(19) United States
(12) Patent Application Publication
(10) Pub. No.: US 2010/0254917 A1
(43) Pub. Date: Oct. 7, 2010

(54) ORGANIC NANO-PARTICLES AND PROCESS FOR THEIR PREPARATION

(76) Inventors: Matthias Jozef Gertruda Brouns, Kerkrade (NL); Joseph Petronella Friederichs, Wessem (NL); Johan Franz Gradus Antonius Jansen, Geleen (NL); Marco Marcus Matheus Driessen, Maasbracht (NL)

Correspondence Address: NIXON & VANDERHYE, PC, 901 NORTH GLEBE ROAD, 11TH FLOOR, ARLINGTON, VA, 22203, US

(21) Appl. No.: 12/668,732

(22) PCT Filed: Jul. 11, 2008
(86) PCT No.: PCT/EP2008/059071

§ 371 Date: Jun. 19, 2010

(30) Foreign Application Priority Data
Jul. 12, 2007 (EP) PCT/EP2007/006187
Jan. 9, 2008 (EP) 08000296.7

(51) Int. Cl. A61K 8/06; A61K 47/32; A61Q 11/00; A61Q 17/04; A61Q 1/00; A61Q 17/02; A61Q 5/00; A61Q 90/00; A61P 17/10; C08L 33/04; G03G 9/08; B32B 27/10

(52) U.S. Cl. 424/55; 424/60; 424/64; 424/70.11; 514/772.6; 524/529; 430/108.4; 428/513; 977/788

(57) ABSTRACT

There is disclosed a process for preparing organic nano-particles comprising the steps of: (a) preparing a solution comprising an unsaturated polyester and/or a vinyl ester resin and a hydrophobic monomer; (b) dispersing the solution obtained in step (a) in an aqueous phase to form a stable emulsion where the aqueous phase is the continuous phase; and (c) using an initiator to cure the emulsion obtained in step (b) in the presence of a water soluble inhibitor to form a dispersion of nano-particles. The invention further provides organic nano-particles obtainable by the process according to the invention; various uses of said nano-particles; such as in paper, dye, toner and personal care compositions and topical medicaments comprising said nano-particles.

The present invention relates to a process for preparing organic nano-particles; the nano-particles obtained and/or obtainable by the process; the use of the nano-particles in the different applications described herein (for example as a plastic pigment for paper coatings, in topical medicaments, and/or in personal care compositions, such as hair care formulations); compositions and formulations comprising the nano-particles and substrates (such as paper) coated with them.

An object of the present invention is to provide improved organic nano-particles. In preferred aspects of the invention, nano-particles of the invention may exhibit tuneable and/or adjustable properties. For example the stability of their shape at high temperature and/or the degree to which they form films when used as a coating or used to prepare paper, may be adjusted.

Another object of the invention is to provide an improved process for making such nano-particles. In one preferred aspect of the invention, the process may be more versatile and provide a more predictable outcome.

Various polymer emulsion have been described in the prior art.

GB 1327111 (Dow) describes water in resin emulsions in which water is the dispersed phase. Dow discloses that the water must be readily removable from the emulsion, so the resins are low density and porous (as the resin is the continuous phase). This teaches away from the hydrophobic nano-particles of the present invention.

WO 2000/73361 and U.S. Pat. No. 6,242,528 (both Eastman Chemical) both describes similar emulsions containing uralkyd and/or acrylic modified alkyd resins as the dispersed phase (which is not a solution). These emulsions are designed to be using as coatings where final curing occurs after the formulation has been applied onto a substrate to form a polymeric coat thereon. This is very different from the emulsions described herein where the very different monomers in the dispersed phase are designed to be (substantially) completely cured whilst still dispersed in the latex (to form dispersed polymeric particles) and where excessive film forming properties are undesired.

GB 1380044 (ICI) describes the preparation of vesicular resins that contain water. The resin may be a copolymer of an unsaturated polyester and an unsaturated monomer such as stryene or methyl methacrylate (MMA). However these resins are formed from a water-in oil (w-o) emulsion where the resin is the continuous phase and the resin particles contain water. This reference also teaches that the resin may also be dispersed in water to form a water-in-oil in water (w-o-w) emulsion (i.e. the dispersed phase is itself a w-o emulsion). However this reference teaches away from forming polymeric particles in which the dispersed phase is a homogenous hydrophobic solution and the continuous phase is aqueous (an oil in water (o-w) emulsion). The resins particles formed by the ICI process would be much larger (5 to 50 microns with vesicles of >0.1 microns, see column 4, lines 18 to 19) and have very different properties to those of the present invention.

GB 1405923 (De Soto Inc) describes aqueous dispersions of acrylic copolymers, designed to coat substrates and form high gloss polymeric films thereon after curing. Thus for example De Soto teaches it is desirable to add an organic solvent to the aqueous dispersion to assist coalescence of the dispersed copolymer particles when the coating is applied (see lines 69 to 77).

DE 1720852 (W. R. Grace) describes a copolymer of vinyl monomers and unsaturated polyester dispersed in water, however there is no teaching of curing o-w emulsion where the dispersed phase is solution of unsaturated polyester or vinyl ester resin with a hydrophobic monomer using the combination of a water soluble inhibitor and an initiator.

WO 2003-093367 (Zhang) describes a nano-composite prepared by polycondensation of an organic polyester resin in the presence of inorganic nano-sized materials. These composite materials typically comprise organic and inorganic domains and are very different from the organic nano-particles of the present invention.

WO 2005-045525 (DSM) describes a mixture of monomers some polymerised cationically and some radically, with relevant initiators and an inorganic filler of particle size 3 to 50 nm. Again the materials obtained are nano-composites with organic and inorganic domains and are completely different the from the organic nano-particles of the present invention.

U.S. Pat. No. 5,416,159 (Imprex) describes liquid polymeric sealants for impregnating porous metal articles to exclude air or oxygen. The sealants comprise 15 to 60% of water dispersible unsaturated polyester the remainder being various different types of acrylate monomer. These compositions are not used in an emulsion polymerization process but are used directly uncured as a sealant composition.

There are deficiencies in all these prior art processes or compositions. Surprisingly, it has now been found that some or all objects of the invention described herein can be achieved by a particular multi-step process and the organic particles so obtained may have useful properties in a wide variety of applications.

Broadly according to the present invention there is provided a process for preparing organic nano-particles comprising the steps of:
(a) preparing a solution comprising an unsaturated polyester and/or a vinyl ester resin and a hydrophobic monomer;
(b) dispersing the hydrophobic solution obtained in step (a) in an aqueous phase to form a stable emulsion where the aqueous phase is the continuous phase; and
(c) using an initiator to cure the (oil in water, o/w) emulsion obtained in step (b) in the presence of a water soluble inhibitor to form a dispersion of nano-particles.

A further optional step (d) comprises collecting the nano-particles from the dispersion obtained in step (c) optionally by drying them at a low temperature (preferably from 20° C. to 60° C., more preferably from 25° C. to 50° C. for example about 45° C.).

Broadly another aspect of the present invention provides organic nano-particles obtained and/or obtainable, preferably obtained, by the process of the invention, optionally in step (d).

Unless the context clearly indicates otherwise, as used herein plural forms of the terms herein are to be construed as including the singular form and vice versa.

The term “comprising” as used herein will be understood to mean that the list following is non-exhaustive and may or may not include any other additional suitable items, for example one or more further feature(s), component(s), ingredient(s) and/or substituent(s) as appropriate.

The terms ‘effective’, ‘acceptable’ ‘active’ and/or ‘suitable’ (for example with reference to any process, use, method, application, preparation, product, material, formulation, compound, monomer, oligomer, polymer precursor, and/or polymers of the present invention and/or described herein as appropriate will be understood to refer to those features of the invention which if used in the correct manner provide the required properties to that which they are added and/or incorporated to be of utility as described herein. Such utility may be direct for example where a material has the required properties for the aforementioned uses and/or indirect for example where a material has use as a synthetic intermediate and/or diagnostic tool in preparing other materials of direct utility. As used herein these terms also denote that a functional group is compatible with producing effective, acceptable, active and/or suitable end products.

As used herein the term ‘nano’ or ‘nano-sized’ denotes at least one linear dimension having a mean size between about 0.1 nm (nm=nanometre=1×10⁻⁷ m) to about 1000 nm. A preferred mean size for the nano-sized materials described herein is less than about 500 nm, more preferably less than about 400 nm most preferably less than about 250 nm, for example less that about 150 nm. Nano-sized materials exist with the nano-size in three dimensions (nano-particles), two dimensions (nano-tubes having a nano sized cross section, but indeterminate length) or one dimension (nano-layers having a nano-sized thickness, but indeterminate area). Usefully the present invention relates to materials that comprise nano-particles.

As used herein the term ‘micro’ or ‘micro-sized’ denotes at least one linear dimension having a mean size above about 1 μm (=1 micron=1×10⁻⁶ m). A preferred mean size for the micro-sized materials described herein is from about 1 to about 100 microns, more preferably from about 1 to about 50 microns, most preferably from about 1 to about 20 nm, for example from about 1 to about 10 microns. Micro-sized materials exist with the micro-size in three dimensions (micro-particles), two dimensions (micro-tubes having a micro-sized cross section, but indeterminate length) or one dimension (micro-layers having a micro-sized thickness, but indeterminate area). Usefully the present invention relates to materials that comprise micro-particles.

Pigments are widely used in paper production to improve the brightness, opacity and printability of the paper to be produced. The major pigment used in the paper industry is calcium carbonate, which has the disadvantage that its properties can not easily be adjusted to meet particular paper requirements, because of the limits of present grinding techniques. To address this problem it has been proposed to use polymer pigments in paper. The polymer pigments that have been proposed so far have, however, the disadvantage that they form films when subjected to pressure in an aqueous environment.

In one embodiment of the present invention the organic nano-particles of the invention may be useful as a colorant (preferably pigment) in paper applications. This is because the process allows the stability of the nano-particles' shape at high temperature to be adjusted according to the conditions used when the selected paper is prepared.

The nano-particles may also be agglomerated to form micro-particles, which have a high pore volume, and thus a low density, which makes them very attractive for various other applications such as, fillers in composite materials which may for example be used in the automotive industry. Other applications and end uses of the nano-particles of the invention are described herein.

Particles of the invention may be useful as an agent to reduce shrinking of composite materials or coatings (especially for materials with a resin based on polyester and/or vinyl ester polymers). The cured nano-particles or micro-particles do not shrink during curing of the composite or coating in use, yet they maintain their other properties, such as thermal expansion and chemical properties. The particles may also be used as gloss or matting agents in coatings (e.g. for paper coating or treatment). The ability of the particles to effect gloss or matt values may be adjusted by selecting the type of resin and monomers as well as by adjusting particle size and cross link density.

The process of the invention uses a solution that may be prepared by dissolving unsaturated polyester and/or a vinyl ester resin in the hydrophobic monomer. The solution may comprise further components, which may be dissolved, dispersed and/or suspended therein. Examples of further components are any suitable: initiators; dyes; pigments; conductive material, such as metal particles; additives, such as emulsifiers, surfactants; small organic compounds, such as hydrophilic monomer; fillers, such as inert inorganic or organic particles and/or cross linkers, such as organic compounds with more than one functional group capable of reacting with vinyl-type double bonds. However, in a preferred embodiment, the solution consists of unsaturated polyester and/or vinyl ester resin and hydrophobic monomer.

Hydrophobic monomer(s) that may be suitable for use in the present invention may be selected from the group consisting of: aromatic (vinyl) compounds, methacrylates and acrylates. The term hydrophobic monomer as used herein encompasses traditional monomers and other compounds with a molecular weight smaller than 500 g/mole that are capable of reacting with the unsaturated polyester and/or vinyl ester resin to form a cross linked network upon curing, and also mixtures comprising at least two of such species whether as a blend of different component or as different functional groups on the same compound.

Preferably the hydrophobic (meth)acrylate comprises C_{>4}hydrocarbo (meth)acrylate(s) and conveniently the C_{>4}hydrocarbo moiety may be C₄₋₂₀hydrocarbyl, more conveniently C₄₋₁₄alkyl most conveniently C₄₋₁₀alkyl, for example C₄₋₈alkyl.

Suitable hydrophobic (meth)acrylate(s) may be selected from: butyl methacrylate, cyclohexyl acrylate, cyclohexyl methacrylate, 2-ethylhexyl acrylate, isoamyl acrylate, isodecyl acrylate, isodecyl methacrylate, isononyl acrylate, isooctyl acrylate, lauryl acrylate, lauryl methacrylate, 2-methylbutyl acrylate, 4-methyl-2-pentyl acrylate, n-butyl acrylate, nonylphenol acrylate, phenoxy methacrylate, phenoxy ethyl methacrylate, sec-butyl acrylate, t-butyl acrylate, and/or mixtures thereof.

Other suitable hydrophobic monomers may be allyl compounds, such as diallyl phtalate, isodecyl allyl ether; vinyl ethers such as butyl vinyl ether and lauryl vinyl ether, as well as other similar monomers and/or mixtures thereof.

Usefully the hydrophobic monomer is an aromatic (vinyl) compound, more usefully an aromatic vinyl monomer, and most usefully an arylalkylene. Convenient arylalkylenes are hydrocarbylenyl substituted benzene, more conveniently C₁₋₁₀alkenyl benzene, most conveniently C₁₋₄alkylenylbenzene and for example monomers selected from: styrene, α-methyl styrene, vinyl toluene, divinyl benzene, t-butyl styrene, di-methyl styrene and/or mixtures thereof, especially styrene.

The hydrophobic monomer may is present in the solution prepared in step (a) of the process of the invention, in an amount from at least 50%, more usefully from 70 to 95% by total weight of the solution, especially if the monomer is a styrene or styrene derivative. Styrene is advantageous due to its low cost and because it forms highly durable nano-particles.

As styrene is environmentally undesirable, where possible its amount should be limited. Hence, in another embodiment of the invention, at the start of step (b), (i.e. just before the solution is mixed with the aqueous phase to form an emulsion) the solution comprises less than 40%, preferably 10 to 30%, of styrene by total weight of the solution. Lowering the amount of styrene has the further advantage of reducing or even eliminating the release of un-reacted styrene in the final product, and thus reducing the undesirable odour of styrene.

However for applications where the environmental effects are less of a concern, in another embodiment, the amount of styrene in the solution at the start of step (b) may be from 20 to 70%, advantageously from 30 to 60%, by weight, which allows for a wide range of properties in the final product.

In addition to hydrophobic monomer(s), hydrophilic monomers may also be present in the solution, in a lower amount by weight than the amount of hydrophobic monomer.

Preferred hydrophilic monomers comprise at least one ethylenically unsaturated carboxylic acid optionally substituted with at least one hydroxyl group. More preferred acids comprise one ethylenic group and one or two carboxy groups, such as C₁₋₄hydrocarbo acrylic acids. Most preferred acid(s) are selected from the group consisting of: acrylic acid (and oligomers thereof), beta carboxy ethyl acrylate, citraconic acid, crotonic acid, fumaric acid, itaconic acid, maleic acid, hydroxyl functional acrylates, methacrylic acid and mixtures thereof. Particular examples of acids may be selected from the group consisting of: acrylic acid (AA), methacrylic acid (MAA), hydroxyethylacrylate (HEA), hydroxyethylmethacrylate (HEMA) and mixtures thereof.

To prevent extended curing in the water phase usually such hydrophilic monomers will be present in an amount of less than 10% by weight, based on total amount of solution prepared in step (a). Without wishing to be bound by any mechanism it is believed that the hydrophilic monomer reduces the tendency of bridging flocculation in step (b) and thus leads to a more stable emulsion.

The terms unsaturated polyester and/or vinyl ester resin used herein denote a polyester having at least one carbon-carbon double bond capable of undergoing radical polymerisation, a vinyl ester having at least one carbon-carbon double bond capable of undergoing radical polymerization; or a (physical or co-polymerized) mixture of unsaturated polyester and unsaturated vinyl ester having at least one carbon-carbon double bond per resin molecule capable of undergoing radical polymerisation.

Preferably the unsaturated polyester and/or the vinyl ester resin have a number average molecular weight per reactive unsaturated group (unsaturation) from 250 to 2500 g/mol and more preferably from 500 to 1500 g/mol. To enhance formation of larger polymer molecules during curing, it is preferred that the unsaturated polyester and/or vinyl ester resin has at least 1 reactive unsaturation group per molecule in which case it is desirable to add a cross linker to enhance formation of a three dimensional polymer network. In a highly advantageous embodiment, the unsaturated polyester and/or vinyl ester resin has an average of at least 1.5 reactive unsaturations per molecule as this leads to organic nano-particles with a well cross linked composition. Highly cross-linked, and hence relatively rigid, nano-particles may be obtained when the unsaturated polyester and/or vinyl ester resin has an average of at least 2.0 reactive unsaturated groups per molecule. For improved control of the curing process it is preferred that the average number of reactive unsaturated groups per molecule is less than 5.0. It has been found that by varying the cross link density, the stability of particle shape at high temperature can be adjusted from relatively soft (using low cross link densities) to relatively rigid (using high cross link densities).

In a further embodiment of the present invention, the unsaturated polyester and/or the vinyl ester resin may have an acid value from 0 to 200 mg KOH/g resin, preferably from 1 to 200 mg KOH/g resin, and more preferably from 10 to 50 mg KOH/g resin. Most preferably the unsaturated polyester resin, where present, has an acid value from 10 to 50 mg KOH/g resin and the vinyl ester resin, where present, has an acid value from 0 to 10 mg KOH/g resin.

The average molecular weight of the unsaturated polyester and/or the vinyl ester resins used in the present invention is preferably from 250 to 5000 g/mol. Without wishing to be bound by any theory the applicant believes that resins of a lower molecular weight do not easily form a cross linked network, whereas resins of a higher molecular weight form very large micelles (and hence larger sized particles) which are harder to stabilise. More preferred unsaturated polyester and/or vinyl ester resins have an average molecular weight from 500 to 4000 g/mol, as it is believed that this leads to a solution of relatively low viscosity and yet allows fast build up of molecular weight during curing.

Preferably the respective weight ratio of the total amount of the unsaturated polyester and/or the vinyl ester resin (component A) to the total amount of hydrophobic monomer (component B) in the solution in step (a) is from 95/5 to 30/70 (ratio A/B), more preferably from 80/20 to 40/60, and most preferably from 75/25 to 50/50. It is believed that when the ingredients are used in these proportions this leads to a superior balance between hydrophilic and hydrophobic properties of the solution and hence the emulsion obtained after step (b) has advantageous properties.

Preferably, the resin solution obtained in step (a) is substantially free from a solvent other than the hydrophobic monomer. The term solvent is used herein to denote an organic solvent and hence the solution may comprise water, although this is not preferred. The term ‘substantially free’ denotes that the content of solvent is less than 1% by weight of the solution. More preferably solvent is present in less than 0.1% by weight of the solution and most preferably the solution has no solvent. A solvent free process has the advantage of forming nano-particles that are much less likely to form films.

Although a mixture of an unsaturated polyester and vinyl ester resin can be used, preferably only one of these compound types will be used.

The aqueous phase used in step (b) of the process of the present invention is preferably a continuous aqueous phase. The aqueous phase may comprise hydrophilic organic compounds, such as alcohol, for example methanol, ethanol, propanol or butanol; DMF, DMSO, organic or inorganic salts. A preferred aqueous continuous phase comprises a base with a pKa of at least 10 in an amount effective to neutralize at least part of the terminal acid groups of the unsaturated polyester and/or vinyl ester resin in the solution which will be added in step (b). It is more preferred that the base is present in an amount to obtain an emulsion with a pH of 3 to 10, as this improves control of the particle size of the nano-particles. Adjusting the emulsion pH to a value of from 6 to 8 is particularly advantageous. Without wishing to be bound by any theory the applicant believes that, improved control of particle size may result from improved control of the polarity of the dispersed droplets in the emulsion which also controls the size at which the droplets are stable. Unless the context indicates otherwise the pH of the aqueous continuous phase is measured by a pH meter (Probe Mettler-Toledo Inpro 200/Pt1000—also used for temperature measurements) the sensor of which is inserted directly into the emulsion.

Surprisingly it has been found, that when the strong base is added during the process strongly influences the outcome of the process. Preferred strong bases have a pKa of at least 10. For example adding the base after the solution has been added to the aqueous phase, forms a much more stable emulsion and substantially less gel forms in the aqueous phase. The curing process can thus be more easily controlled with improved results.

The amount of the base needed may be calculated from the acid value of the solution prepared in step (a). Examples of suitable bases include KOH, NaOH, ammonia and triethylamine, and if any of these bases are used the solution prepared in step (a) will easily form an emulsion.

In yet another embodiment, at least one emulsifier is added prior to and/or during step (b) to enhance emulsification. The emulsifiers may be cationic emulsifiers, anionic emulsifiers and/or non-ionic emulsifiers. Examples of suitable emulsifiers (also referred to as surfactants) are listed in “Applied Surfactants—principles and application” by Tharwat F. Tadros, (2005), John Wiley and Sons Ltd, incorporated herein by reference. However, emulsifiers are costly and any residual amount in the final product represents a safety and/or health issue in certain applications, such as packaging of food or medicaments. It will therefore be appreciated that the process of the present invention may be conducted without emulsifier being added.

The emulsions prepared during the process of the invention are oil in water (o-w) emulsions as discrete droplets of the solution prepared in step (a) (which is homogenous and hydrophobic) are stably dispersed in an aqueous continuous phase (the water). This is to be contrasted with water in oil (w-o) emulsions where a hydrophobic (organic) phase is the continuous phase and water droplets are dispersed therein. Emulsion polymerisation in an o-w emulsion can be better controlled to produce a desired size of nano-particle, since particle size corresponds to the size of the droplet and o-w emulsions are more readily stabilised over a wider range of droplet sizes. An o-w emulsion has the further advantage that during exothermal curing, the reaction temperature can be more easily controlled as the (aqueous) continuous phase is a better thermal conductor.

The droplet size of the hydrophobic solution in the aqueous emulsion may be from about 5 nm to 1000 nm, advantageously 10 nm to 500 nm, more advantageously 50 nm to 400 nm, most advantageously 50 nm to 250 nm. As used herein, unless the specified otherwise, droplet size in an emulsion refers to their average diameter as established by laser diffraction (using a Beckman-Coulter LS230).

The amount of water to be used in step (b) will depend on the desired solids content, as well as on the amount of the base to be used. In general, high solids content is advantageous as this leads to better process control and less waste. However, since the water also acts as a temperature buffer during the exothermal curing process, it is preferred that the organic phase of the emulsion is not continuous. If a dye or pigment is present, very high solids content may be obtained whereas if no dye or pigment is present, then the emulsions may be stable with a solids content of about 10 to 40% by weight. Advantageous emulsions have a solids content of 10 to 60%, more advantageously 20 to 40% by weight. Surprisingly cured emulsions having a solids content of 20 to 40% by weight were also stable. The term ‘stable emulsion’ as used herein denotes that the emulsion does not show phase separation for two hours after preparation.

Step (b) may be performed at a temperature from 10 to 100° C., preferably from 15 to 90° C., and/or over a period of time from 30 minutes to 48 hours, preferably from 1 hour to 4 hours.

In step (b) the solution prepared in step (a) can be emulsified by adding it to the stirred aqueous phase by any suitable means such as mechanical mixing, ultrasound and/or any other method well known by skilled person for preparing emulsions. The mixing may be simple stirring or high shear mixing.

In a preferred embodiment, the unsaturated polymer is an unsaturated polyester. Highly acid functional unsaturated polyesters are preferred, as these provide high acid values, which facilitate emulsification. Preferably, the unsaturated polyester is substantially linear. The term ‘substantially linear’ as used herein in denotes that at least 80% by weight of the polymer is in the backbone of the polymer. Preferably, the unsaturated polyester is multi-unsaturated, where the average number of unsaturations per molecule is greater than 1.

Examples of suitable unsaturated polyester or vinyl ester resins that can be used in the present invention are subdivided in the categories as classified by Malik et al. in J. M. S.—Rev. Macromol. Chem. Phys., C40(2&3), p. 139-165 (2000) the contents of which is hereby incorporated herein by reference. These polymers include:
(1) Ortho-resins, based on phtalic anhydride, maleic anhydride, or fumaric acid and glycols, such as 1,2-propylene glycol, ethylene glycol, diethylene glycol, triethylene glycol, 1,3-propylene glycol, dipropylene glycol, tripropylene glycol, neopentyl glycol and/or hydrogenated bisphenol-A. Common ortho-resins are derived from 1,2-propylene glycol are used in combination with a reactive diluent such as styrene.
(2) Iso-resins, prepared from isophtalic acid, maleic anhydride or fumaric acid, and glycols. These resins may contain higher proportions of reactive diluent than the ortho resins.
(3) Bisphenol-A-fumarates, based on ethoxylated bisphenol-A and fumaric acid.
(4) Chlorendic resins, prepared from chlorine/bromine containing anhydrides or phenols used in the preparation of unsaturated polyester resins.
(5) Vinyl ester resins, with unsaturated sites in the terminal position only, introduced by reacting epoxy resins with (meth)acrylic acid and/or (meth)acrylamide. Suitable epoxy resins may comprise diglycidyl ethers of bisphenol-A, epoxies of the phenol-novolac type, and/or epoxies based on tetrabromobisphenol-A. Vinyl ester resins are mostly used because of their hydrolytic resistance, excellent mechanical properties, and low emission of styrene.
(6) Dicyclopentadiene (DCPD) resins, analogues thereof and vinyl ester urethanes. DCPD-resins and their analogues may for example be obtained either by modification of any of the above resin types by Diels-Alder reaction with cyclopentadiene, and/or by first reacting maleic acid with dicyclopentadiene, followed by resin manufacture as indicated above.

The above resins may be modified according to methods known to the person skilled in the art, for example by adjusting (e.g. lowering) acid number, hydroxyl number or anhydride number, or increasing flexibility by inserting flexible units in the polymer backbone.

Other reactive groups that are curable by a radical reaction may also be present in the resins described herein (such as the unsaturated polyester and/or vinyl ester resin used in the present invention). Such reactive groups may be derived from itaconic acid, citraconic acid and allylic groups.

Unsaturated polyester and/or vinyl esters resins are advantageous in providing more acid stable nano-particles.

The unsaturated polyester resins and/or vinyl ester resins to be used in accordance with the present invention may be any of the above types of resins or a mixture of two or more of these resins. Preferably, however, they are chosen from the group consisting of iso-phtalic resins and ortho-phtalic resins and vinyl ester resins.

More preferably, the resin is an unsaturated polyester resin chosen from the group consisting of DCPD-resins, iso-phthalic resins and ortho-phtalic resins, as these types have the highest acid values.

The unsaturated polyester resins and/or vinyl ester resins of use in the present invention contain reactive unsaturations, i.e. unsaturations which are capable of undergoing a radical (co)polymerisation, and they may in addition contain unreactive unsaturations like the aromatic ring in phtalic anhydride.

The unsaturated polyester resins or vinyl ester resins of use in the present invention may contain solvents. The solvents may be inert to the resin system or may be reactive therewith during the curing step.

Hydrophobic monomers are required for the invention and act as a reactive diluent. Suitable hydrophobic monomers include those already described herein, for example aromatic vinyl compounds like styrene, α-methyl styrene, divinyl benzene; methacrylates like: t-butyl methacrylate, cyclohexyl methacrylate, phenoxy methacrylate, phenoxy ethyl methacrylate, lauryl methacrylate; acrylates like t-butyl acrylate, nonylphenol acrylate, cyclohexyl acrylate, lauryl acrylate, isodecyl acrylate, isobornyl acrylate; allyl compounds like diallylphtalate, isodecylallyl ether; vinyl ethers like butyl vinyl ether, lauryl vinyl ether and the like as well as suitable mixtures thereof.

The initiator to be used in the curing process of the present invention can suitably be at least part of an initiator complex. Such an initiator complex can be any radical initiator such as, for instance, azo compounds, persulphates or peroxides. The initiator complex can be a one-component initiator complex in which decomposition is triggered by heat or it can be a two-component initiator complex which is triggered by adding a co-initiator. For both the one and two component initiator complexes it is strongly preferred that at least one of the initiator components is oil soluble. During emulsion polymerisation (curing step (c)) an oil soluble initiator, incorporated into the dispersed hydrophobic phase, can then initiate polymerisation of the unsaturated polyester, vinyl ester and hydrophobic monomer solution prepared in step (a) (and dispersed in water in step (b)).

A water soluble inhibitor may be added at a number of stages in the process (as discussed herein) to ensure that it is present when needed in the curing process. The required concentration of water soluble initiator depends on the specific initiator as well as the concentration and/or composition of the other components of the system and may be determined by standard experimental work by the person skilled in the art.

For azo compounds, it is preferred that the initiator is selected from the group consisting of azobisisobutyronitrile (AIBN), 1,1′-azobis(cyclohexanenitrile), 1,1′-azobis(2,4,4-trimethylpentane) and dicumyl peroxide (DCP).

Preferably the, radical initiator in the initiator complex is a peroxide such as any peroxide known to the skilled person as suitable for curing of unsaturated polyester resins or vinyl ester resins. Such peroxides comprise organic and inorganic peroxides, whether solid or liquid. Examples of suitable peroxides comprise: hydrogen peroxide, peroxy carbonates (comprising at least one —O(C═O)O— moiety), peroxyesters (comprising at least one —(C═O)OO— moiety), diacylperoxides (comprising at least one —(C═O)OO(C═O)— moiety), and/or dialkylperoxides (comprising at least one —OO— moiety). Suitable peroxides can also be oligomeric or polymeric in nature. Examples of other suitable peroxides are well known to those skilled in the art and for example are described in US 2002/0091214-A1, paragraph [0018] which is hereby incorporated herein by reference.

Preferred peroxides are organic, example of which comprise: tertiary alkyl hydroperoxides (such as t-butyl hydroperoxide), other hydroperoxides (such as cumene hydroperoxide), ketone peroxides (a special class of hydroperoxides formed as the addition product of hydrogen peroxide and a ketone, examples of which include methyl ethyl ketone peroxide, methyl isobutyl ketone peroxide and acetylacetone peroxide), peroxyesters or peracids (such as t-butyl peresters, benzoyl peroxide, peracetates and perbenzoates, lauryl peroxide, (including (di)peroxyesters)); and/or perethers (such as, for instance, peroxy diethyl ether).

Commonly used organic peroxides curing agents comprise tertiary peresters or tertiary hydroperoxides, that is peroxy compounds having tertiary carbon atoms directly attached to an —OO(C═O)OH or —OOH group. Mixtures of two or more different peroxides (such as any of those described herein) may be used in the process of the present invention. The peroxides may also be multi-peroxide functional, i.e. they may comprise any two or more different peroxygen bearing moieties in one molecule. If a solid peroxide is used as the curing agent, it is preferably benzoyl peroxide (BPO). Ketone peroxides are a more preferred class of peroxides, methyl ethyl ketone peroxide (MEK peroxide) being the most preferred as it is cheap and easy to handle. Preferably at least one part of the initiator complex is selected from the group consisting of peranhydrides, peresters and hydroperoxides (including perketones).

The initiator used to cure the emulsion may advantageously be added to the aqueous phase before step (b), to allow the initiator to be well dispersed before emulsification. Optionally if the initiator is added before step (b) it is added at relatively low temperature to prevent premature curing of the solution before emulsification.

As an alternative, initiator may be added during the emulsification step (b) so it will be uniformly distributed throughout the mixture as the emulsion is formed (e.g. by mechanical stirring).

In another option the initiator may be added after the emulsion has been formed in step (b), which has the advantageous in that premature curing of the system is prevented.

Initiator may also be added in various amounts at more than one stage during the process.

The same initiator(s) may also behave differently depending at what point in the process they are added. For example MEKP and a copper compound may be used differently in the process of the present invention. In one option using MEKP and a copper salt (e.g. copper acetate), the MEKP may be added to the hydrophobic solution (so the MEKP will be located within the particles) and curing may start from the waterphase by adding the copper ion (as part of the salt which is water soluble). Alternatively for example MEKP and another copper compound (copper napthanate) may be used, in which case the copper napthanate may be added to the solution (so it is located within the particles) and curing may be started from the water phase by adding MEKP (or any other suitable water soluble initiator).

Preferably step (a) of the process of the present invention is performed at a temperature of 10 to 100° C., more preferably from 20 to 50° C., most preferably at ambient temperature (25° C.±5°).

In a preferred embodiment of the present invention, the pH of the aqueous emulsion obtained in step (b), after formation of the polymer-based nano-particles, is from 3 to 11, more preferably from 6 to 8.

The curing of the aqueous emulsion obtained in step (b) strongly depends on the type of initiator complex used.

When a single component initiator complex is used in step (a), curing in step (c) may be achieved by heat. If the initiator complex is activated thermally, the temperature of the aqueous emulsion from step (b) can be gradually increased to the desired temperature, for instance by heating the emulsion slowly to a temperature of 70° C. over three hours.

As well as thermally activated initiator complexes, redox initiators can be used in step (c). For example an aromatic amine can be dissolved with benzoyl peroxide in the unsaturated polyester and/or vinyl ester resin or added later.

During the curing of the emulsified solution prepared in step (b), the emulsion should contain a water soluble inhibitor. In other words, a water soluble inhibitor must be present during the curing of the emulsified solution at a concentration that prevents extensive curing in the aqueous phase in step (c) thus eliminating or strongly reducing flocculation. The water soluble inhibitor may be added at a number of stages in the process, as discussed herein. The required concentration depends on the choice of water soluble inhibitor as well as the concentration and composition of the other components of the system and may be determined by standard experimental work by the person skilled in the art.

Surprisingly it was found that inhibiting the curing reaction in the water phase and hence preventing or at least greatly reduce bridging flocculation in the water phase leads to improved process control and a more uniform and predictable outcome of the process. Without wishing to be bound by any theory, the applicant believes the presence of an effective amount of water soluble inhibitor may allow emulsions to be prepared with much higher solids content than was previously obtainable.

Surprisingly, it was also found that having a water soluble inhibitor present in the emulsion during curing was particularly advantageous for curing at elevated temperatures, such as 50 to 90° C., more preferably at 60 to 80° C. An inhibitor that is soluble in the solution prepared in step (a) may be added in a suitable amount to inhibit polymerisation from occurring immediately to ensure that the curing process will only start after step (b) has been initiated or completed.

The term ‘water soluble inhibitor’ is used herein to denote any suitable ingredient that can inhibit polymerisation in an aqueous medium, such as a water soluble chain terminator, and/or a water miscible or water soluble chain transfer agent. The water soluble inhibitor may be added at one or more of stages of the process of the invention each of which has different advantages.

In a preferred embodiment, the water soluble inhibitor is added in the aqueous phase before emulsification in step (b). This allows for direct preparation of the aqueous phase and the water soluble inhibitor does not need to migrate from the solution into the aqueous phase to inhibit curing.

In another embodiment, the water soluble inhibitor is added to the solution before step (b) to increase the chances that premature curing in the solution is prevented before the inhibitor is dispersed in the water phase.

In yet another embodiment, the water soluble inhibitor is added to the mixture during step (b) as the solution is being dispersed in the water phase. This allows for a more uniform distribution of the inhibitor as it is more intimately admixed with the other ingredients as the emulsion is prepared.

The inhibitor also may be added after step (b), after the emulsion has been formed. It is then preferred that the emulsion is formed at low temperature to prevent premature curing in the water phase before the inhibitor is present.

Inhibitors, which are not water soluble, i.e. which are at least partially dissolvable in an organic phase, may also be used in the process of the invention, but such inhibitors are used in addition to water soluble inhibitors. Solvent soluble inhibitors may for example be used to preventing premature curing in the solution before it is dispersed in the aqueous phase.

In step (a) of the present invention other ingredients may be added to the solution of the unsaturated polyester and the monomer such as (i) a catalyst and (ii) an inhibitor for inhibiting at least part of the polymerisation of the unsaturated polyester and the monomer during steps (a) and (b). A preferred solution prepared in step (a) may thus also contain one or more inhibitors, more preferably chosen from the group consisting of: phenolic compounds, stable radicals such as galvinoxyl and N-oxyl based compounds, catechols and/or phenothiazines.

The amount of inhibitor added to the solution prepared in step (a) may vary within wide ranges. One parameter that may be used to select the amount of inhibitor is to indicate the amount of gel that is desired to be achieved. For example if a phenolic inhibitor added to the solution in step (a), preferably it is added in an amount that results in gel being formed at the end of step (a) in an amount from about 0.001 to 35 mmol per kg of the solution, more preferably from about 0.01 to 35 mmol per kg, and most preferably from about 0.1 to 35 mmol per kg. The skilled person can easily assess, for each inhibitor selected, the amount that will needed to give good results in the process of the invention.

Suitable examples of inhibitors that can be used in the solution of step (a) may be selected from the group consisting of: 2-methoxyphenol, 4-methoxyphenol, 2,6-di-t-butyl-4-methylphenol, 2,6-di-t-butylphenol, 2,4,6-trimethyl-phenol, 2,4,6-tris-dimethylaminomethyl phenol, 4,4′-thio-bis(3-methyl-6-t-butylphenol), 4,4′-isopropylidene diphenol, 2,4-di-t-butylphenol, 6,6′-di-t-butyl-2,2′-methylene di-p-cresol, hydroquinone, 2-methylhydroquinone, 2-t-butylhydroquinone, 2,5-di-t-butylhydroquinone, 2,6-di-t-butylhydroquinone, 2,6-dimethylhydroquinone, 2,3,5-trimethylhydroquinone, catechol, 4-t-butylcatechol, 4,6-di-t-butylcatechol, benzoquinone, 2,3,5,6-tetrachloro-1,4-benzoquinone, methylbenzoquinone, 2,6-dimethylbenzoquinone, napthoquinone, 1-oxyl-2,2,6,6-tetramethylpiperidine, 1-oxyl-2,2,6,6-tetramethylpiperidine-4-ol (also referred to herein as TEMPOL), 1-oxyl-2,2,6,6-tetramethylpiperidine-4-one (also referred to herein as TEMPON), 1-oxyl-2,2,6,6-tetramethyl-4-carboxyl-piperidine (also referred to as 4-carboxy-TEMPO), 1-oxyl-2,2,6,6-tetramethyl-3-carboxyl-piperidine (also referred to as 3-carboxy-TEMPO), 1-oxyl-2,2,5,5-tetramethylpyrrolidine, 1-oxyl-2,2,5,5-tetramethyl-3-carboxylpyrrolidine (also referred to herein as 3-carboxy-PROXYL), aluminium-N-nitrosophenyl hydroxylamine (also referred to herein as cupferron), diethylhydroxylamine, phenothiazine, gallic acid, propyl gallate Fremy's salt (disodium and/or dipotassium salt(s) of nitrosodisulfonic acid), derivatives thereof, isomers thereof, salts thereof, combinations of any of these in the same species and/or any mixtures thereof.

Optionally the inhibitor is a chain transfer agent, such as mercapto ethanol, mercapto acetic acid, mercapto propionic acid, derivatives thereof, isomers thereof, salts thereof, combinations of any of these in the same species and/or any mixtures thereof. The chain transfer agents may be both solvent soluble inhibitors and water soluble inhibitors if as they are soluble and/or miscible in both these liquid types.

Solvent soluble inhibitors are particularly advantageous when an activator (also referred to as a promoter) is present in the solution prior to emulsifying of the solution in the water phase. In this case, the inhibitor is preferably added to the solution prior to addition of the activator/promoter to ensure that substantial curing does not take place until the emulsification has taken place, i.e. during steps (a) and (b). Typically, the inhibitor is used or degrades/reacts during step (a) and/or (b) so that the curing reaction is initiated when the inhibitor concentration decreases to below a threshold value. Hence, a solvent soluble inhibitor is suitably present in an effective amount to inhibit polymerisation during steps (a) and (b).

The effect of water soluble and solvent soluble inhibitors may be completely different, and hence both types may advantageously be present in the emulsion, particularly if an activator is used in the organic phase.

Preferred water soluble inhibitors are selected from the group consisting of chain terminators: such as gallic acid, propyl gallate, TEMPOL, TEMPON, 3-carboxy TEMPO, carboxy-PROPYL, Cupferron, Fremy's salt, derivatives thereof, isomers thereof, salts thereof, combinations thereof in the same species and/or mixtures thereof; and chain transfer agents: such as mercapto ethanol, mercapto acetic acid, mercapto propionic acid derivatives thereof, isomers thereof, salts thereof, combinations thereof in the same species and/or mixtures thereof.

The catalyst which may be used in step (a) can suitably be a tertiary aromatic amine selected from the group consisting of dimethylaniline, dimethyltoluidine, 4-tertiary-butyl-N,N-dimethylaniline, 4-methoxy-dimethylaniline, diethylaniline, diethyl-toluidine, N,N-diisopropylaniline, diisopropyltoluidine, dimethylolaniline, dimethylol-toluidine, N,N-diethanolaniline, N,N-diethanoltoluidine, N,N-diethanolaniline mono-methylether, N,N-diethanolaniline dimethylether, N,N-diisopropanolaniline, N,N-diisopropanoltoluidine, N,N-diisopropanoltoluidine monomethyl ether, N,N-diisopropanoltoluidine dimethyl ether, N,N,N′,N′-tetramethylbenzidine, 4,4′-methylene-bis(2,6-diisopropyl-N,N-dimethylaniline), 4,4′-vinylidene-bis(N,N-dimethylaniline), N,N-digly-cidyl-4-glycidyloxyaniline, N,N-diglycidylaniline, 4-dimethylaminophenethyl alcohol, 4,4-methylene-bis(N,N-bis-glycidylaniline). Also ethoxylated or propoxylated anilines, respectively ethoxylated or propoxylated toluidines may be used. Preferably said amine compound is chosen from the group of aromatic tertiary amines having a β-hydroxy or a β-alkoxy (generally C₁₋₁₂) substituent. Suitable examples of aromatic tertiary amines, and of β-hydroxy- or β-alkoxy-substituted aromatic tertiary amines are listed herein.

One or more catalysts may be used in accordance with the present invention. Suitably, the amount of catalyst in the solution in step (a) is in the range of from 0.01 to 10% by weight, based on the total weight of the solution prepared in step (a). More preferably, the amount of inhibitor in the solution prepared in step (a) is in the range of from 0.1 to 2% by weight, based on the total weight of the solution prepared in step (a).

It is strongly preferred that curing takes place in the emulsion, i.e. when the solution is dispersed in the water, as this leads to superior control of the particle size of the resulting nano-particles. Thus it is desirable that curing should take place before the emulsion is applied to a substrate (for example as a coating). Without wishing to be bound by any theory, it is believed that during curing, the unsaturated polyester and/or vinyl ester resin react with the hydrophobic monomers, to form a rigid cross linked polymeric network within each droplet or micelle. In step (c) curing may be achieved by increasing the temperature of the aqueous emulsion obtained in step (b) to from 30 to 100° C., preferably from 70 to 90° C.

Step (b) (emulsification) can be performed at room-temperature after which the emulsion may be heated to 30 to 100° C., preferably to 70 to 90° C. There will be an optimum curing temperature which balances curing time and curing speed and will depend one the one-component initiator complex used and factors such as its decomposition temperature. The optimum temperature may change if an inhibitor is used as this has a strong impact on the gel time.

Step (b) can be carried out in the absence or presence of an additional emulsifier, although for the reasons already given herein it is preferred not to use additional emulsifier if not necessary.

In the process according to the present invention, steps (b) and (c) take place over a total period of from 0.5 hours to 48 hours.

The term ‘curing’ as used herein with respect to step (c) of the invention means the process of forming cross links between the molecules of the unsaturated polyester and/or vinyl ester resin and the hydrophobic monomer. Curing takes place while the solution forms a dispersed (hydrophobic) phase with the aqueous continuous phase of the emulsion so discrete nano-particles are formed. If the solution is allowed to dry to form an (uncured) coating or precipitates are allowed to form prior to curing, then curing the mixture will not form nano-particles.

According to another embodiment of the invention, the curing step is performed by adding both components of a two-component initiation complex to the solution prepared in step (a). Preferably, both components of the initiator complex are oil soluble. In this case the use of inhibitors (as described herein), are essential as they postpone the start of the curing process to allow time to prepare the emulsion. Preferred two-component systems are peresters or peranhydrides as one component with tertiary aromatic amines as the second component, or hydroperoxides (such as perketones) as one component with a transition metal as the second component. Alternatively, one of the components may be a persulphate and/or an azo compound.

Suitable transition metal components are transition metal salts or complexes and may be selected from the group consisting of: salts and/or complexes of: cobalt, vanadium, manganese, copper and/or iron and/or mixtures thereof. The transition metal salts or complexes can be water soluble or oil soluble. Preferred oil soluble transition metal salts comprise transition metals carboxylates such as C₆₋₂₀ carboxylates for example 2-ethyl hexanoates, octanoates, and isodecanoates. The transition metal salt or complex may be present in amount of at least 0.05 mmol per kg of resin solution, more preferably at least 1 mmol per kg. The upper limit of the transition metal content is not very critical, although to save money very high concentrations are not used. Generally, the concentration of the transition metal salt or complex added to the solution prepared in step (a) will be less than 50 mmol per kg of the solution, preferably less than 20 mmol per kg. Copper salts and complexes are especially preferred.

In yet another embodiment of the invention, the curing process is started by adding the second component of a two-component initiator complex. This embodiment is especially preferred when one of the components of the two-component initiator complex is a water soluble component. Examples of a suitable two-component initiator complex comprise: an oil soluble transition metal salt or complex as the first component with a water soluble peroxide (such as hydrogen peroxide) as the second component: or an oil soluble peroxide as the first component with a water soluble transition metal salt or complex as the second component. Examples of water soluble transition metal salts are the chlorides, bromides, iodides, acetates and lactates of the transition metals described herein.

The present invention also relates to organic nano-particles obtained and/or obtainable by the process of the present invention. The organic nano-particles display such a wide variety of properties such as stability, strength, porosity, and low density, that they are suitable for use in many applications such as those described herein. One example of a suitable use is in automotive applications, where traditionally heavy metal parts are used. The nano-particles of the present invention may be prepared so they are stable up to 200° C. This property ensures that these particles do not form films if they are used as a plastic pigment to manufacture paper.

Organic nano-particles of the present invention may be prepared with an average particle size diameter (as measured by laser diffraction (Beckman-Coulter LS230)) of from 10 nm to 1,000 nm. The largest nano-particles within this range (and even larger homogeneous micro-particles up to 10 microns) may be prepared without adding base so the emulsion comprises much larger water droplets and high energy mixing is required to prepare a stable emulsion. Micro-sized particles prepared in the absence of base may be different from those prepared as described herein by agglomeration of nano-particles (for example those micro-particles prepared without agglomeration may have a more regular (e.g. spheroid) shape). Preferred nano-particles have an average particle size from 20 to 500 nm and more preferably from 50 to 150 nm.

An aspect of the present invention further provides the use of the organic nano-particles of the present invention as plastic pigment, preferably as a plastic pigment for coating substrates. A yet other aspect of the present invention provides a substrate coated with a coating comprising nano-particles of the present invention. Preferred substrates are thin flexible substrates, more preferably absorbent substrates, most preferably fibrous substrates, for example substrates comprising non-woven fibres, such as paper.

In a still further aspect of the present invention there is provided a process for preparing organic micro-particles by subjecting organic nano-particles obtained and/or obtainable by the process of the present invention to spray-drying, freeze-drying, coagulation, flocculation and/or agglomeration (or any other analogous process), and recovering the organic micro-particles so formed. Agglomeration may for example be achieved by increasing pH or evaporating water or a solvent.

An aspect of the present invention provides for organic micro-particles that are obtained and/or obtainable by the process of the present invention as described herein. Organic micro-particles of the invention display unique properties in terms of stability, strength, porosity, and thus low density. Preferred organic micro-particles have an average particle size (measured as described herein) of from 500 nm (0.5 microns=5×10⁻⁷ m) to 100 microns (100 μm=1×10⁻⁴ m), more preferably from 1 to 10 microns.

The nano-particles and/or micro-particles of the present invention may be isolated from the emulsion and collected, and yet have the advantageous property that they can be readily be re-dispersed in water to form a stable aqueous emulsion.

It will further be appreciated that in the process of the invention that other ingredients (such as those described herein) can be added first to the polymer solution and then to the aqueous phase or vice versa, first to the aqueous phase and then to the polymer solution. The solution and aqueous phase may also be mixed in any suitable manner for example by dosing the aqueous phase with the solution or vice versa.

Some features of the invention are illustrated non exhaustively by the examples. For example compositions of the invention can be produced with high solids content. Many different resins can be used with redox initiators (and only some of such resins that are suitable are exemplified herein). Many alternative bases can be used. It has been found that gallic acid is a particularly preferred initiator for use in the process of the invention.

The present invention also relates to the use of the organic micro-particles obtained and/or obtainable by the present process in a sheet moulding compound.

In addition, the present invention relates to the use of the present organic nano-particles and/or micro particles for encapsulating particles of a colorant composition, and to colorant compositions comprising the organic nano-particles and/or micro-particles of the present invention. Colorant denotes any coloured material (including materials which absorb or reflect UV or IR radiation instead of or in addition to visible light) and includes dyes and pigments. Dyes are generally soluble in the medium to which they are added and are typically (but not exclusively) organic liquids. Pigments are generally insoluble in the medium to which they are added and typically (but not exclusively) inorganic solids. Preferably the colorant is a dye.

Encapsulating of particles of colorant composition may take place suspending particles of colorant composition in the solution prior to emulsification or during emulsification, so that particles of colorant composition is encapsulated in the nano-particles during curing of the solution. Alternatively, the particles of colorant composition may be added after the curing reaction, so that the encapsulation takes place during the optional agglomeration process.

Further, the present invention relates to the use of the present organic nano-particles and/or micro-particles as binder for a toner composition, and to toner compositions comprising the organic nano-particles and/or micro-particles in accordance with the present invention.

A non-limiting list of other end uses for the nano-particles and/or the micro-particles of the invention include:
a) As additives for sheet moulded compounds (also denoted herein as SMC), where the presence of the particles (particularly spray dried particles) leads to lower density products;
b) As plastic pigment, particularly for coatings, such as paper coatings, where the presence of the particles may provide high gloss or tuneable gloss properties of the product;
c) As fillers in composite materials and particularly in concrete, where for example the use of the particles as micro fillers may increase strength, lower porosity, reduce density and/or prevent water penetration into the structure;
d) As filler for coatings, where for example the particles may provide anti blocking properties to the coating, increase scratch resistance, lower abrasion, increase drying speed, reduce the required amount of solvent, and reduce shrink;
e) As filler for waxes, where for example the particles may provide a lubricating effect, reduce weight, reduce abrasion and/or act as a high temperature filler;
f) Mono-disperse particles of the invention may be used for spacers for example in display applications,
g) As hybrid colorant, where for example a colorant (preferably pigment) particle may interact with the particles of the invention in several ways. In one option pigment particles may be dispersed in the emulsion prior to curing of the resin and monomer. The pigment particles (which are usually hydrophobic) will tend to migrate inside the hydrophobic droplets of the solution. Curing the solution will form particles in which a pigment core is encapsulated within a shell of cured polymer. In another option the pigment particles may be co-agglomerated with the nano-particles to form micro-particles. Both methods can produce pigments which are dispersible in water and which can be either partially accessible or inaccessible for direct contact with ambient atmosphere or other materials.
h) In adhesives, where for example the particles may be used as a filler or as a shrink reducing agent, since the particles will be inert during curing of the adhesive. As the particles will be strongly connected to the adhesive they will have no detrimental effect on adhesive strength.
i) As encapsulating agent for active ingredients that may be added to the emulsion prior to curing and remain in the particle upon curing. The resulting particles, which contain the active ingredient, are more easily dispersible and the active ingredient is protected. Examples of active ingredients are dyes and UV-blockers.
j) As an ingredient in compositions suitable for use in personal care and/or as topical medicaments.

It will be appreciated that some of the above uses a) to j) may overlap.

Depending on the desired properties of the topical medicament and/or personal care composition, particles of the invention (organic nano-particles and/or micro-particles) may be present in an amount from 0.001 to 99%, preferably 0.1 to 80%, more preferably 0.5 to 50%, most preferably 1 to 20% by weight of the total composition. Any suitable, conventional ingredients suitable for such applications may be used, such as those well known to a skilled formulator of such compositions.

Topical medicament indicates a composition which is formulated for the delivery of a therapeutically active agent to, or via, the skin. A wide variety of active agents may be delivered using such formulations, including agents that are intended for treatment of the skin, such as anti-acne agents, and systemically-active agents for which the skin is merely the route of administration, rather than the site of action.

Non limiting examples of personal care compositions (which may or may not be applied topically) include: cosmetic compositions, hair-care products, insect repellents, oral hygiene compositions, self-tanning products, sunscreens, toiletry compositions, mixtures thereof, and/or combinations in the same composition.

Cosmetic composition indicates a composition that may be used on the body to modify its appearance. Non limiting examples of such compositions may include: after-sun compositions, blushers, colour cosmetics, eye shadows, face creams, face masks, foundations, lip balms, lipsticks, moisturisers, powder formulations, temporary tattoos and other forms of body art; and toner cleansers. Cosmetic compositions may be applied as any suitable formulation type, non limiting examples of which include: creams, dispersions, emulsions (such as water in oil (w-o), oil in water (o-w), water in oil in water (w-o-w) and oil in water in oil (o-w-o), although for the reasons already given emulsions where the continuous phase is aqueous such as o-w and w-o-w are preferred), gels, lotions, milks, ointments, pastes, powders, roll-on, salves, serums, solutions, spray, sticks and suspensions.

Hair care composition indicates a composition that may be used on animal hair, preferably on human hair, most preferably on the human head. Non limiting examples of such a composition may include suitable: conditioners, creams, foams, gels, hair dyes, hair colorants, hair styling products, hot oil treatments, lotions, mascaras, masks, mousses, muds, rinses, shampoos, styling sprays and/or waxes.

Oral hygiene composition indicates a composition that may be suitable for use in oral hygiene, dental treatment and/or be otherwise applied to the buccal and/or oral cavity. Non limiting examples of such a composition may include suitable: chewing gums, dentifrices, denture cleansing formulations, flosses, glass ionomer cements, lozenges, mouth sprays, mouthwashes, tooth paints, tooth pastes and/or toothpowders.

Sunscreen indicates a composition that may be used on the body to provide protection against the sun's rays or other UV sources. Non limiting examples of such compositions may also include: sun blockers and/or tanning lotions.

Toiletry composition indicates a composition that may be used on the body to clean, scour, wash, perfume and/or reduce odour. Non limiting examples of such a composition may include suitable: anti-microbial compositions, bath products (e.g. bath foams and bath salts), deodorants, detergents, perfumes, soaps and/or shower gels.

Many other variations embodiments of the invention will be apparent to those skilled in the art and such variations are contemplated within the broad scope of the present invention.

Further aspects of the invention, preferred features and embodiments thereof are given in the claims herein.

### EXAMPLES

The present invention will now be described in detail with reference to the following non limiting examples which are by way of illustration only. The following abbreviations have been used:
Cu-napthanate denotes Copper Napthanate available commercially from Rockwood pigments under the trade designation Nuodex copper 8;
BPO denotes benzoyl peroxide available commercially from Akzo Nobel under the trade designation Perkadox CH 50L;
DMPT denotes the accelerator dimethyl-p-toluidine;
DTBC denotes the inhibitor di-tertiary butyl catechol;
KPS denotes potassium peroxo disulphate also known as potassium persulphate;
LPO denotes lauryl peroxide available commercially from Aldrich under the trade designation Luperox LP
MY denotes the dye methyl yellow (CAS 60-11-7),
MEKP denotes the redox initiator methyl ethyl ketone peroxide available commercially from Bentley Chemicals under the trade designation Butanox M50;
UP denotes an unsaturated polyester;
UPS1 denotes the composite resin comprising UP and styrene in respective weight ratio of 65 to 35 and available commercially from DSM under the registered trade mark Palatal® P6-01); UPS2 denotes the composite resin comprising UP and styrene in respective weight ratio of 64 to 36 and available commercially from DSM under the registered trade mark Palatal® P4-01);
UPS3 denotes the composite resin comprising UP and styrene in respective weight ratio of 64 to 36 and available commercially from DSM under the registered trade mark Palatal® P5-01);
UPS4 denotes the composite resin comprising UP and styrene in respective weight ratio of 65 to 35 and available commercially from DSM under the registered trade mark Palapreg® P 0432-02);

The reactor in the syntheses described herein is a 1 litre baffled, glass lined reactor equipped with mechanical stainless steel stirrer and fitted with a reflux condenser, a droplet funnel and a Mettler-Toledo Iwo 200/Pt 1000 probe for pH and temperature measurements. The reactor is also provided with a Lauda type K6Ks external heating/cooling system to control reactor temperature.

Unless otherwise indicated herein particle size distribution (also denoted PSD) of the exemplified particles of the invention is based on the diameter of the particles and is determined by laser diffraction (using a Beckman-Coulter LS230) of the particles dispersed within a latex as described below.

### Example 1

### Synthesis of Nano-Particles

Procedure: Thermal Initiation from the Organic Phase (Initiator BPO)

BPO initiator (8.1 g) is dissolved in 270 g of a solution of UP and styrene (UPS1) to produce a clear solution. Water (580 g) is added to the reactor and then stirred at 650 rpm whilst the solution of initiator and UPS1 is added over 15 minutes at room temperature. This produces an emulsion of the solution of initiator and UPS1 dispersed in water, the solids content being about 30% by weight.

KOH (8.1 g) and gallic acid (10 mg) are dissolved in 50 ml water and added to the reactor drop by drop over a period of 10 to 15 minutes until the mixture reaches a pH of 6, when the stability of the emulsion increases substantially.

After 5 minutes of further stirring at approximately 650 rpm, the temperature is raised to 70° C. and a stable emulsion results. The emulsion is allowed to cure thermally whilst it is continuously stirred for a further 6 hours at 70° C. to obtain a stable latex of suspended, cross linked nano-particles in a water phase.

PSD is from the minimum size detectable (about 40 nm) up to about 400 nm with a D₅₀ of 120 to 130 nm.

Example 1 can be used as an ingredient in a typical hair care composition such as the shampoo described in Example 15.

### Example 2

### Heat Drying of Suspension

A sample of the suspension from Example 1 is slowly dried at 40° C. in air in a low temperature oven to form a powder with a glass transition temperature (T_{g}) of 130° C. as determined by differential thermal calorimetry (DTC). The powder was observed up to 200° C. and exhibited no tacky behaviour or other effects such as melt, flow or degradation that are typical of film formation.

### Example 3

### Spray Drying of Suspension

A further example is prepared both to scale up the process used above and to form micro particles. A dispersion prepared as described in Example 1 with about 30% by weight solids, is spray dried using a standard pilot plant spray dryer set with the following parameters: nozzle 0.34 mm, 70 bar air pressure, 180° C. in spray tower, throughput 200 g/min.

The spay drier produces about 45% by weight of a tower fraction of rough particles and about 55% by weight of a cyclone fraction of finer particles. The two fractions are combined to form a free flowing dry powder of micro-particles consisting of hollow spheres of strongly bonded agglomerates of nano-particles. The micro-particles have sizes of 1 to 20 microns (PSD of the dry dispersion based on linear diameter and measured using laser diffraction with a Rodos dispersing unit available commercially from Sympatec Helos.)

This examples shows the nano-particles of Example 1 may be agglomerated into micro particles using standard equipment and processing.

### Comparative Example 4

### Synthesis of Nano-Particles

Procedure: Initiation from the Water Phase (Thermal Initiator: Water Soluble KPS):

KPS initiator (8.1 g) is dissolved in water (50 g) to form an initiator solution which is added to the reactor. Further water (530 g) is added to the reactor and then stirred at 650 rpm whilst a solution (270 g) of UP dissolved in styrene (UPS1) is added over 15 minutes at room temperature. This produces an emulsion of the UPS1 dispersed in water.

KOH (8.1 g) and gallic acid (10 mg) are dissolved in 50 ml water and added to the reactor drop by drop over a period of 10 to 15 minutes until the mixture reaches a pH of about 6.

After 5 minutes of further stirring at approximately 650 rpm, the temperature of the mixture is raised to 70° C. to initiate curing. However the system coagulates during the curing process, as the emulsion is not stable.

Without wishing to be bound by any theory it is believed the instability is both due to the water soluble initiator causing flocculation in the water phase during curing and the high solids content (30% by weight) of the emulsion. This shows that if a water soluble initiation system is used, the emulsion is difficult to stabilise during curing and further measures, such as adding an emulsifier to the mixture, are required. Therefore water insoluble initiators are preferred, particularly if the emulsion has a high solids content.

### Comparative Example 5

### Synthesis of Nano-Particles

Procedure: No Temperature Control (Thermal Initiator. BPO):

An example is prepared using a similar procedure to that described in Example 1, except that during curing, the temperature is allowed to increase with the heat due to the exothermal curing reaction. The system coagulates at 85° C., as the emulsion is unstable.

Without wishing to be bound by any theory it is believed the instability may be caused by a combination of the high temperature and the high solids content (about 30% by weight) of the emulsion. To keep the emulsion stable the temperature should be kept lower than the coagulation temperature particularly during curing. Coagulation temperature depends on the properties of the system used, such as composition and concentration, but may be easily be determined by a person skilled in the art, for example by preparing this comparative example.

### Comparative Example 6

### Synthesis of Nano-Particles

### Procedure: No pH Restrictions (Thermal Initiator: BPO):

An example is prepared using a similar procedure to that described in Example 1, except that that prior to heating and curing the KOH/gallic acid solution is added until the pH of the mixture reaches 11. After heating the mixture to 70° C. the emulsion is unstable and the system coagulates within a few minutes.

Without wishing to be bound by any theory it is believed the instability may be caused by a combination of the high pH and the high solids content (about 30% by weight) of the emulsion. To keep the emulsion stable the pH should be kept lower than the coagulation pH at all times. Coagulation pH depends on the properties of the system used, such as composition and concentration, but may be easily be determined by a person skilled in the art.

### Comparative Example 7

### Synthesis of Nano-Particles

Procedure: No pH Restrictions. Adding the Base Before the UP (Thermal Initiator BPO)

An example is prepared using a similar procedure to that described in Example 1, except that the solution of KOH and gallic acid is added to the water until a pH of about 11.5 is reached and before the solution of initiator/UP/styrene is added to the reactor. When the stirred mixture is heated to about 70° C. to start curing the emulsion destabilises after 5 to 60 minutes and in many cases whilst the solution is being added to the reactor.

Comparative examples 4 and 5 show that dispersions with a high solids content may usefully be prepared under controlled pH conditions to prevent coagulation. Without wishing to be bound by any theory it is believed that keeping the pH of the present example (Comparative Example 7) below 7.5 may be particularly important as this may be a critical boundary. The exact value of the critical pH may depend strongly on the system, such as chemical composition, temperature, solid content and presence of optional emulsifier. Adding a base to the reactor before the solution of initiator, UP and stryene may produce more extreme effects, as temporary pH values up to 12 will be reached, often resulting in immediate destabilisation.

### Example 8

### Synthesis of Nano-Particles

Procedure: Redox Initiation. BPO

BPO initiator (8.1 g) and DTBC inhibitor (100 ppm) are dissolved in 270 g of a solution of UP/styrene (UPS1) to produce a clear solution. Water (580 g) is added to the reactor. DMPT accelerator (4 g) is added to the solution of initiator and UPS1, and the resultant solution is added to the reactor over 15 minutes at room temperature whilst the mixture is stirred at 650 rpm. This produces an emulsion of the solution of initiator and UPS1 dispersed in water, the solids content being about 30% by weight.

KOH (8.1 g) and gallic acid (10 mg) are dissolved in 50 ml water and added to the reactor drop by drop over a period of 10 to 15 minutes until the mixture reaches a pH of 6, when the stability of the emulsion increases substantially.

The emulsion is allowed to cure (free radical curing) whilst it is continuously stirred for a further 6 hours at room temperature (25° C.) to obtain a stable latex of suspended, cross linked nano-particles in a water phase.

PSD is from the minimum size detectable (about 40 nm) up to about 250 nm with a D₅₀ of 90 to 95 nm.

It is possible to prepare nano-particles of the invention using a redox initiation system, showing the versatility of the process of the invention.

### Example 9

### Synthesis of Nano-Particles

### Procedure: Redox Initiation. (Initiator: MEKP):

MEKP (8.1 g) is dissolved in 270 g of a solution of UP/styrene (UPS2) to produce a clear solution. Water (580 g) is added to the reactor which is stirred at 650 rpm whilst the solution of initiator and UPS2 is added to the reactor over 15 minutes at room temperature. This produces an emulsion of the solution of initiator and UPS2 dispersed in water, the solids content being about 30% by weight.

KOH (8.1 g) and gallic acid (10 mg) are dissolved in 50 ml water and added to the reactor drop by drop over a period of 10 to 15 minutes until the mixture reaches a pH of 6, when the stability of the emulsion increases substantially.

Stirring of the mixture continues whilst copper acetate (0.02% by weight, based on the total solids content) is added to initiate curing of the UPS2. The emulsion is allowed to cure (free radical curing) whilst it is continuously stirred for a further 6 hours at room temperature (25° C.) to obtain a stable latex of suspended, cross linked nano-particles in a water phase.

PSD is from the minimum size detectable (about 40 nm) up to about 180 nm with a D₅₀ of 90 to 95 nm.

### Example 10

### Heat Drying of Suspension

A sample of the suspension from Example 9 is slowly dried at 40° C. in air in a low temperature oven resulting in a transparent brittle film. The resins UPS2 (used in Example 10) has a lower concentration of unsaturated groups (degree of unsaturation) than the resins UPS1 (used in Example 1). Without wishing to be bound by any theory it is believed that the particles obtained in Example 10 may thus be less rigid than those of Example 1.

It can be seen that varying the composition of the resins in the solution and/or the process conditions can be used to adjust the properties of the nano-particles obtained as desired (for example tune their degree of film formation).

### Example 11

### Synthesis of Nano-Particles

### Procedure: Redox Initiation: (Initiator: MEKP)

MEKP (8.1 g) is dissolved in 270 g of a solution of UP/styrene (UPS3) to produce a clear solution. Water (580 g) is added to the reactor and stirred at 650 rpm whilst the solution of initiator and UPS3 is added to the reactor over 15 minutes at room temperature. This produces an emulsion of the solution of initiator and UPS3 dispersed in water, the solids content being about 30% by weight.

KOH (8.1 g) and gallic acid (10 mg) are dissolved in 50 ml water and added to the reactor drop by drop over a period of 10 to 15 minutes until the mixture reaches a pH of 6, when the stability of the emulsion increases substantially.

Stirring of the mixture continues for five minutes and the reactor is heated to 45° C. Copper acetate (0.02% by weight, based on the total solids content) is then added to the stirred mixture to initiate curing of the UPS3. The emulsion is allowed to cure (free radical curing) whilst it is continuously stirred for a further 8 hours 45° C. to obtain a stable latex of suspended, cross linked nano-particles in a water phase.

PSD is from the minimum size detectable (about 40 nm) up to about 300 nm with a D₅₀ of 125 nm.

### Example 12

### Synthesis of Nano-Particles

### Procedure: Redox Initiation: (Initiator: MEKP)

MEKP (8.1 g) is dissolved in 270 g of a solution of UP/styrene (UPS1) to produce a clear solution. Water (580 g) is added to the reactor and stirred at 650 rpm whilst the solution of initiator and UPS1 is added to the reactor over 15 minutes at room temperature. This produces an emulsion of the solution of initiator and UPS1 dispersed in water, the solids content being about 30% by weight.

KOH (8.8 g) and gallic acid (10 mg) are dissolved in 50 ml water and added to the reactor drop by drop over a period of 10 to 15 minutes until the mixture reaches a pH of 6, when the stability of the emulsion increases substantially.

Copper acetate (0.02% by weight, based on the total solids content) is then added to the stirred mixture to initiate curing of the UPS1. The emulsion is allowed to cure (free radical curing) whilst it is continuously stirred for a further 4 hours at room temperature to obtain a stable latex of suspended, cross linked nano-particles in a water phase. In order to increase styrene conversion a post cure step is performed for an additional 2 hr at 60° C.

PSD is from the minimum size detectable (about 40 nm) up to about 300 nm with a D₅₀ of 105 nm.

A comparison of Examples 11 and 12 shows that different reins can be used with redox initiators.

### Example 13

### Synthesis of Nano-Particles

### Procedure: Redox Initiation: (Initiator: MEKP) Change Charge Order of Chemicals.

270 g of a solution of UP/styrene (UPS1) is added to the reactor. MEKP (8.1 g) is added to the reactor and dissolved within the UP/styrene mixture. KOH (8.8 g) and Gallic acid (10 mg) are dissolved in 630 ml water and added to the reactor drop by drop over a period of 45 to 60 minutes at room temperature. After an additional 1 hr of stabilisation this method produces an emulsion of the solution of initiator and UPS1 dispersed in water, the solids content being about 30% by weight.

Copper acetate (0.02% by weight, based on the total solids content) is then added to the stirred mixture to initiate curing of the UPS1. The emulsion is allowed to cure (free radical curing) whilst it is continuously stirred for a further 4 hours 25° C. to obtain a stable latex of suspended, cross linked nano-particles in a water phase. To increase the styrene conversion a post-cure step is performed for an additional 2 hrs at 60° C.

PSD is from the minimum size detectable (about 40 nm) up to about 300 nm with a D₅₀ of 125 nm.

A comparison of Examples 12 and 13 shows that ingredients can be added in a different order and at different doses and stable emulsions of cross linked particles may be obtained.

### Example 14

### Synthesis of Nano-Particles

### Procedure: Redox Initiation: (Initiator: MEKP), Use of Alternative Base.

270 g of a solution of UP/styrene (UPS1) is added to the reactor. 12.6 g Tri-Ethylene-Amine (TEA) is added to the solution and dissolved whilst stirring. Next MEKP (8.1 g) is added to the reactor and dissolved within the UP/styrene/TEA mixture. Gallic acid (10 mg) is dissolved in 630 ml water and this solution is added to the reactor drop by drop over a period of 45 to 60 minutes at room temperature. After an additional 1 hr of stabilisation this method produces an emulsion of the solution of initiator and UPS1 dispersed in water, the solids content being about 30% by weight.

Copper acetate (0.02% by weight, based on the total solids content) is then added to the stirred mixture to initiate curing of the UPS1. The emulsion is allowed to cure (free radical curing) whilst it is continuously stirred for a further 4 hours 25° C. to obtain a stable latex of suspended, cross linked nano-particles in a water phase. To increase the styrene conversion a post-cure step is performed for an additional 2 hrs.

PSD is from the minimum size detectable (about 40 nm) up to about 300 nm with a D₅₀ of 125 nm.

A comparison of Examples 12 and 14 shows that different bases can be used in the neutralization process, and stable emulsions of cross linked particles may be obtained.

### Comparative Example 15

### Synthesis of Nano-Particles

### Procedure: Redox Initiation: in the Absence of a Waterphase Inhibitor.

MEKP (8.1 g) is dissolved in 270 g of a solution of UP/styrene (UPS1) to produce a clear solution. Water (580 g) is added to the reactor and stirred at 650 rpm whilst the solution of initiator and UPS1 is added to the reactor over 15 minutes at room temperature. This produces an emulsion of the solution of initiator and UPS1 dispersed in water, the solids content being about 30% by weight.

KOH (8.8 g) is dissolved in 50 ml water and added to the reactor drop by drop over a period of 10 to 15 minutes until the mixture reaches a pH of 6, when the stability of the emulsion increases substantially.

Copper acetate (0.02% by weight, based on the total solids content) is then added to the stirred mixture to initiate curing of the UPS1. The emulsion is allowed to cure (free radical curing) whilst it is continuously stirred for a further 4 hours 25° C. to obtain a stable latex of suspended, cross linked nano-particles in a water phase. To increase the styrene conversion a post-cure step is performed for an additional 2 hrs at 60° C.

PSD is from the minimum size detectable (about 40 nm) up to about 1000 nm with a D₅₀ of 179 nm.

A comparison of Examples 12 and 15 shows that if a water phase inhibitor is not used, the PSD broadens and the D₅₀ increases.

### Example 16

### Synthesis of Nano-Particles

### Procedure: Redox Initiation: (Initiator: MEKP) High Solids.

TEA (1.15 g) and Cu-napthanate (0.13 g) are dissolved in 40 g of a solution of UP/styrene (UPS4) to produce a clear solution. Water (58 g) containing 0.2 g of an additional surfactant (potassium salt of Sartomer SMA 1000P) is added to a 200 ml beaker equipped with a magnetic stirrer. The solution of TEA, Cu-napthanate and UPS4 is added to the beaker over 15 minutes at room temperature whilst stirring. This produces an emulsion of the solution of Cu-napthanate and UPS4 dispersed in water, the solids content being about 40% by weight. Subsequently MEKP (1.2 g) is added to the stirred mixture to initiate curing of the UPS4. After approx 4 hours at room temperature a stable dispersion is obtained. D₅₀ of the particles being 100 nm.

Example 16 shows that the process of the invention can be used to obtain dispersions with a high solids content and low average particle size.

### Example 17

### Dye Encapsulation

### Procedure: Thermal Initiation

LPO initiator (4.5 g) and MY (0.5 g) are dissolved in 280 g of a solution of UP and styrene (UPS1) to produce a yellow solution. Water (670 g) is added to the reactor and then stirred at 650 rpm whilst the solution of initiator, dye and UPS1 is added over 15 minutes at room temperature. This produces an emulsion of the solution of initiator, and UPS1 dispersed in water, the solids content being about 30% by weight.

KOH (8.1 g) and gallic acid (10 mg) are dissolved in 50 ml water and added to the reactor drop by drop over a period of 10 to 15 minutes until the mixture reaches a pH of 6, when the stability of the emulsion increases substantially.

After 5 minutes of further stirring at approximately 650 rpm, the temperature is raised to 70° C. and a stable emulsion results. The emulsion is allowed to cure thermally whilst it is continuously stirred for a further 6 hours at 70° C. to obtain a yellow stable latex of suspended, cross linked nano-particles in a water phase.

### Example 18

### Heat Drying of Encapsulated Dye

A sample of the suspension from Example 12 is slowly dried at 40° C. in air in a low temperature oven resulting in a yellow powder, which upon rinsing with water remained yellow. This shows that the dye is incorporated in the nano-particles during the synthesis in Example 12 or in the agglomerates during the drying process.

### Example 19

### Adjustable Gloss

Examples are prepared to demonstrate the effect of the particles of the invention on gloss

The dispersion of Example 1 (30% solids, UPS1) is used to prepare a plastic pigment for each of the coating formulations (1 to 5) described in Table 1 below, in which polyvinyl alcohol (PVA) is used as a binder. The molecular weight of PVA is from 31000 to 50000 g/mole and the degree of hydrolysis 88 to 90%. The amount of binder used is 82% by weight of plastic pigment for each of Coatings 1 to 4 and 60% by weight of plastic pigment for Coating 5.

The formulations are applied to Form HK penetration charts (219 mm×286 mm, available commercially from Leneta) using a K101 control coater (available commercially from RK print coat instruments Ltd.), The coater is set to apply a coating thickness of 50 μm. After coating, the charts are dried at 140° C. for three minutes and the coating gloss is measured at an angle of 85° with a BYK-Gardner micro-Tri-gloss instrument.

TABLE 1
Gloss of Coatings 1 to 5

| | Dispersion | Plastic | | | | Gloss |
|---|---|---|---|---|---|---|
| Coating | Ex 1/g | pigment/g | PVA/g | Water/g | Solids/% | (85°)/% |
| 1 | 5 | 1.5 | 0.33 | 95 | 1.8 | 26.9 |
| 2 | 25 | 7.5 | 1.65 | 75 | 9.0 | 36.0 |
| 3 | 50 | 15.0 | 3.30 | 50 | 17.7 | 46.3 |
| 4 | 75 | 22.5 | 4.95 | 25 | 26.2 | 52.2 |
| 5 | 25 | 7.5 | 4.95 | 75 | 11.8 | 40.5 |

The data in Table 1 show that gloss strongly improves as the amount of the plastic pigment of the invention increases (Coatings 1 to 4). It can been seen (comparing Coating 2 with Coating 5) that tripling the amount of PVA in a formulation produces only a limited increase in gloss (about 4.5 points), whereas tripling the amount of plastic pigment in a formulation (Coating 4 compared to Coating 5) increases the gloss to a much greater extent (about 11.7 points).

It will be appreciated that the gloss of a coated substrate may be adjusted either by changing the amount of nano-particles in the coating or the composition of the nano-particle.

### Example 20

### Personal Care Application

A conventional personal care composition (shampoo formulation) may be prepared by mixing the following ingredients in the given amounts, where Example 1 indicates the isolated nano-particles collected from the latex as prepared in Example 1.

### Example 20

| | Amount |
|---|---|
| Ingredient | (by weight %) |
| Sodium laureth sulfate | 8 |
| Cocamidopropyl betaine | 3 |
| Sodium chloride | 2 |
| Panthenol | 0.5 |
| Propylene glycol | 0.3 |
| Benzophenone-4 | 0.2 |
| Glycerin | 0.2 |
| Phenoxyethanol | 0.2 |
| Alcohol denat. | 0.1 |
| Citric acid | 0.1 |
| Tetrasodium EDTA | 0.02 |
| Water | to 100 |

### Example 21

A topical medicament can be prepared by mixing nano-particles (such as Example 1) with a suitable active ingredient and conventional topical delivery medium.

### Example 22

### Synthesis of Nano-Particles

Procedure: Initiation from the Water Phase with Emulsifier (Thermal Initiator: KPS):

KPS initiator (8.1 g) is dissolved in water (50 g) to form an initiator solution which is added to the reactor. Further water (530 g) is added to the reactor and then stirred at 650 rpm whilst a solution (270 g) of UP dissolved in styrene (UPS1) is added over 15 minutes at room temperature. This produces an emulsion of the UP/styrene solution dispersed in water. A suitable ionic emulsifier (such as 1 g sodium lauryl sulphate, SLS) is added to the mixture to improve its stability in the next step.

KOH (8.1 g) and gallic acid (10 mg) are dissolved in 50 ml water and added to the reactor drop-wise over a period of 10 to 15 minutes until the mixture reaches a pH of about 6.

After 5 minutes of further stirring at approximately 650 rpm, the temperature of the mixture is raised to 70° C. to initiate curing to obtain a stable latex of suspended, cross linked nano-particles in a water phase.

### DISCUSSION

Nano-particles of the invention should be synthesised at temperatures lower than the coagulation temperature of the emulsion. For many of the examples described herein, the coagulation temperature is found to be about 75° C., so the examples are prepared below 75° C. It is found that the maximum temperature of the reaction, particularly the curing step, depends on the concentration of initiator system, monomer and unsaturated polyester and/or vinyl ester resin. Using a water soluble inhibitor system permits higher maximum reaction temperatures to be reached without de-stabilising the emulsion. Of those ingredients tested, gallic acid has been found to stabilise the system to the greatest extent

Nano-particles of the invention should also be synthesised at a pH lower than the coagulation pH of the emulsion. The coagulation pH depends on the properties of the specific system used such as composition and concentration of ingredients. For the many of the examples herein the coagulation pH is found to be about 7.5. The coagulation pH for any given system may easily be established by a person skilled in the art.

1. A process for preparing organic nano-particles comprising the steps of:
(a) preparing a solution comprising an unsaturated polyester and/or a vinyl ester resin and a hydrophobic monomer;
(b) dispersing the solution obtained in step (a) in an aqueous phase to form a stable emulsion where the aqueous phase is the continuous phase; and
(c) using an initiator to cure the emulsion obtained in step (b) in the presence of a water soluble inhibitor to form a dispersion of nano-particles.

2. The process as claimed in claim 1, comprising the further step of:
(d) collecting the nano-particles from the dispersion obtained in step (c) optionally by drying them at a low temperature.

3. The process according to claim 1, where the water soluble inhibitor and/or the initiator is added to the aqueous phase before step (b).

4. The process according to claim 1, where the water soluble inhibitor and/or the initiator is added to the solution before step (b).

5. The process according to claim 1, where the water soluble inhibitor and/or the initiator is added during step (b).

6. The process according to claim 1, where the water soluble inhibitor and/or the initiator is added after step (b).

7. The process according to claim 1, where the hydrophobic monomer is selected from the group consisting of: aromatic (vinyl) compounds, methacrylates and acrylates.

8. The process according to claim 7, where the hydrophobic monomer is an aromatic monomer.

9. The process according to claim 8, where the hydrophobic monomer is a C₁₋₄alkylenylbenzene.

10. The process according to claim 9, where the hydrophobic monomer is selected from the group consisting of: styrene, α-methyl styrene, vinyl toluene, di-vinyl benzene, t-butyl styrene, di-methyl styrene and mixtures thereof.

11. The process according to claim 10, where the hydrophobic monomer is styrene.

12. The process according to claim 1, where the unsaturated polyester and/or the vinyl ester resin has a number average molecular weight per reactive unsaturated group thereon of from 250-2500 g/mol.

13. The process according to claim 12, where the unsaturated polyester and/or vinyl ester resin has at least one reactive unsaturated group per molecule.

14. The process according to claim 13, where the unsaturated polyester and/or vinyl ester resin has an average of at least 1.5 reactive unsaturated groups per molecule.

15. The process according to claim 14, where the unsaturated polyester and/or vinyl ester resin has an average of at least 2.0 reactive unsaturated groups per molecule.

16. The process according to claim 1, where the unsaturated polyester and/or the vinyl ester resin has an acid value from 0 to 200 mg KOH/g resin.

17. The process according to claim 16, where the unsaturated polyester and/or the vinyl ester resin has an acid value from 10 to 50 mg KOH/g resin.

18. The process according to claim 17, where the unsaturated polyester and/or the vinyl ester resin has an acid value from 0 to 10 mg KOH/g resin.

19. The process according to claim 1, where initiator is selected from the group consisting of: persulphates, azo initiators and peroxides, optionally from the group of peroxides consisting of: peranhydrides, peresters and hydroperoxides.

20. The process according to claim 1, where after step (b) at least 90% by weight of the water soluble inhibitor is present in the aqueous continuous phase of the emulsion.

21. The process according to claim 1, where the water soluble inhibitor is selected from the group of consisting of chain terminators, chain transfer agents, derivatives thereof, salts thereof, combinations thereof in the same species, and mixtures thereof.

22. The process according to claim 1, where the water soluble inhibitor is a chain terminator selected from group of consisting of: gallic acid, propyl gallate, TEMPOL, TEMPON, 3-carboxy TEMPO, carboxy-PROPYL, Cupferron, Fremy's salt, derivatives thereof, isomers thereof, combinations thereof in the same species, and mixtures thereof.

23. The process according to claim 1, where the water soluble inhibitor is a chain transfer agent, selected from group of consisting of: mercapto ethanol, mercapto acetic acid, mercapto propionic acid, derivatives thereof, isomers thereof salts thereof, combinations thereof in the same species, and mixtures thereof.

24. The process according to claim 1, where the number average molecular weight of the unsaturated polyester and/or the vinyl ester resin is from 250 to 5000 g/mol.

25. The process according to claim 1, where the respective weight ratio of the unsaturated polyester and/or the vinyl ester resin (component A) and the hydrophobic monomer (component B) in the solution in step (a) is from 95/5 to 40/60.

26. The process according to claim 25, where the respective weight ratio (A to B) is from 80/20 to 40/60.

27. The process according to claim 26, where the respective weight ratio (A to B) is from 75/25 to 50/50.

28. The process according to claim 1, where at least 50% by weight of the hydrophobic monomer is styrene.

29. The process according to claim 28, where from 70 to 95% by weight of the hydrophobic monomer is styrene.

30. The process according to claim 1, where at the start of the emulsification step (b), from 20 to 70% by weight of the solution is styrene.

31. The process according to claim 30, where at the start of the emulsification step (b), from 30 to 60% by weight of the solution is styrene.

32. The process according to claim 1, where no emulsifier is present during emulsification in step (b).

33. The process according to claim 1, comprising the still further step of adding a base with a pKa of at least 10 in an amount effective to neutralize at least part of the terminal acid groups of the unsaturated polyester and/or vinyl ester resin.

34. The process according to claim 33, where the base is added in an amount to adjust the pH of the mixture to which it is added to a value from 3 to 10.

35. The process according to claim 34, where the base is added in an amount to adjust the pH of the mixture to which it is added to a value from 6 to 8.

36. The process according to claim 35, where the base is added in an amount to adjust the pH of the mixture to which it is added to a value from 6 to 7.5.

37. The process according to claim 33, where the base is added after the solution has been added to the aqueous phase.

38. The process according to claim 1, where the pH of the aqueous emulsion obtained in step (b) after the organic nano-particles have been formed is from 5 to 8.

39. The process according to claim 38, where the pH of the aqueous emulsion obtained in step (b) after the organic nano-particles have been formed is from 6 to 8.

40. The process according to claim 1, where the dispersed phase of the emulsion obtained in step (b) has an average droplet size of from 5 nm to 1000 nm.

41. The process according to claim 40, where the dispersed phase of the emulsion obtained in step (b) has an average droplet size of from 50 nm to 400 nm.

42. The process according to claim 1, where the solution prepared in step (a) is substantially free of a solvent other than the hydrophobic monomer.

43. The process according to claim 1, where the cured nano-particles have been agglomerated prior to separation from the emulsion.

44. Organic nano-particles obtained and/or obtainable by the process according to claim 1, where the nano-particles are dispersed as an emulsion in an aqueous phase or separated from the aqueous emulsion.

45. Use of the organic nano-particles according to claim 44 as plastic pigment, preferably as a plastic pigment for paper coating.

46. A non-woven fibrous substrate, preferably paper, comprising a coating comprising nano-particles according to claim 44.

47. A colorant composition, preferably dye composition, comprising organic nano-particles according to claim 44.

48. A toner composition comprising organic nano-particles according to claim 44.

49. A process for preparing organic micro-particles by spray-drying, freeze drying coagulating, agglomerating and/or flocculating the organic nano-particles according to claim 44 and then recovering the organic micro-particles so formed.

50. Organic micro-particles obtainable by the process claimed in claim 49.

51. Use of the organic nano-particles as claimed in claim 44 and/or the organic micro-particles as a ingredient in sheet moulded compounds (SMC); as a plastic pigment; as a filler in composite materials, coatings, concrete, waxes; as a spacer; as a hybrid pigment; as an encapsulating agent (for example for dyes and UV-blockers) and/or as a ingredient in adhesives.

52. Use as an ingredient in personal care compositions and/or topical medicaments of one or more of:
i) organic nano-particles obtained by (a) preparing a solution comprising an unsaturated polyester and/or a vinyl ester resin and a hydrophobic monomer; (b) dispersing the solution obtained in step (a) in an aqueous phase to form a stable emulsion where the aqueous phase is the continuous phase; and (c) using an initiator to cure the emulsion obtained in step (b) in the presence of a water soluble inhibitor to form a dispersion of nano-particles and where the nano-particles are dispersed as an emulsion in an aqueous phase or separated from the aqueous emulsion,
ii) organic micro-particles obtained by (a) preparing a solution comprising an unsaturated polyester and/or a vinyl ester resin and a hydrophobic monomer; (b) dispersing the solution obtained in step (a) in an aqueous phase to form a stable emulsion where the aqueous phase is the continuous phase; and (c) using an initiator to cure the emulsion obtained in step (b) in the presence of a water soluble inhibitor to form a dispersion of nano-particles, wherein the nano-particles are made by spray-drying, freeze drying coagulating, agglomerating and/or flocculating the organic nano-particles and then recovering the organic micro-particles so formed;
iii) organic nano-particles obtained and/or obtainable by a process comprising the steps of: (a) preparing a solution comprising an unsaturated polyester and/or a vinyl ester resin, an initiator and a hydrophobic monomer; (b) emulsifying the solution obtained in step (a) in an aqueous phase; (c) curing the emulsified solution; and/or
iv) organic micro-particles obtained and/or obtainable by spray-drying, freeze drying, coagulating, agglomerating and/or flocculating the organic nano-particles described in component (iii).

53. A personal care composition comprising the organic micro-particles and/or the organic nano-particles as described and/or claimed in claim 44.

54. A personal care composition as claimed in claim 53 selected from the group consisting of cosmetic compositions, hair-care products, insect repellents, oral hygiene compositions, self-tanning products, sunscreens, toiletry compositions and mixtures thereof.

55. A topical medicament comprising the organic micro-particles and/or the organic nano-particles as described and/or claimed in claim 44.
